(19) United States
(12) Patent Application Publication
(10) Pub. No.: US 2025/0339705 A1
(43) Pub. Date: Nov. 6, 2025

(54) Method and Apparatus Using Personalized I-Wave Periodicity Dependent Quadri-Pulse Theta Burst Stimulation (iQPS) To Treat Neurological or Psychiatric Disorders

(71) Applicant: Christian Hirschbeck, Munich (DE)

(72) Inventor: Christian Hirschbeck, Munich (DE)

(21) Appl. No.: 19/198,014

(22) Filed: May 3, 2025

Related U.S. Application Data
(60) Provisional application No. 63/642,694, filed on May 4, 2024.

(51) Int. Cl. A61N 2/00; G16H 20/40

(52) U.S. Cl. CPC A61N 2/006; G16H 20/40

(57) ABSTRACT

The present invention relates to an apparatus for treating neurological or psychiatric disorders using i-wave periodicity-dependent Quadri-Pulse Theta Burst Stimulation (iQPS). The system comprises a transcranial magnetic stimulation (TMS) stimulator configured to deliver test pulses to a target motor cortical area and therapeutic theta bursts to a treatment area with pulse frequency tailored to the patient's individual i-wave periodicity. An electromyography (EMG) device records motor evoked potentials (MEPs) from a target muscle and analyzes the intervals between MEPs to determine the i-wave periodicity. The personalized stimulation enhances cortical plasticity by synchronizing stimulation with the brain's intrinsic neural timing. The invention is applicable to neurological and psychiatric conditions that include depression, schizophrenia, stroke recovery and neuropathic pain. Advantages include improved treatment efficacy, reduced variability in patient response and a non-invasive, adaptive therapy model that aligns with the patient's unique neurophysiological characteristics, offering a precise and scalable approach to brain stimulation.

### CROSS REFERENCE TO RELATED APPLICATION

This disclosure claims the benefit of the priority of U.S. Provisional Patent Application No. 63/642,694, entitled “Method And Apparatus For Using Personalized I-Wave Periodicity Dependent Quadri-Pulse Theta Burst Stimulation (iQPS) To Treat Neurological Or Psychiatric Disorders” and filed on May 4, 2024. The above-identified application is incorporated herein by reference in its entirety.

### TECHNICAL FIELD

The invention generally relates to the field of transcranial magnetic stimulation (TMS), and more specifically to a method and apparatus for using personalized i-wave periodicity dependent Quadri-Pulse Theta Burst Stimulation (iQPS) to treat neurological or psychiatric disorders.

### BACKGROUND

The background description includes information that may be useful in understanding the present invention. It is not an admission that any of the information provided herein is prior art or relevant to the presently claimed invention, or that any publication specifically or implicitly referenced is prior art.

Transcranial magnetic stimulation (TMS) has emerged over the past few decades as a powerful non-invasive technique for modulating cortical excitability and influencing neural circuits in the human brain. Traditionally, TMS has been employed to induce motor-evoked potentials (MEPs) by stimulating motor cortical regions, thereby offering a direct means of assessing and modulating corticospinal excitability. By applying repetitive magnetic pulses to specific areas of the cortex, clinicians and researchers have been able to induce changes in synaptic plasticity, mimicking mechanisms akin to long-term potentiation (LTP) or long-term depression (LTD) which are foundational to learning and memory processes. The plastic changes have been leveraged in the therapeutic management of a variety of neurological and psychiatric disorders including depression, Parkinson's disease and post-stroke rehabilitation.

One of the most widely used approaches in therapeutic TMS is repetitive TMS (rTMS) which involves the delivery of repeated magnetic pulses at specific frequencies. High-frequency rTMS (>5 Hz) has generally been associated with increased cortical excitability while low-frequency rTMS (≤1 Hz) tends to suppress cortical activity. Despite its demonstrated efficacy, conventional rTMS protocols often require lengthy stimulation sessions over several weeks to produce meaningful clinical outcomes. Furthermore, the protocols typically apply the same stimulation parameters across individuals without accounting for inter-individual variability in cortical physiology. The limitation poses a significant challenge as patient responses to rTMS are highly variable leading to inconsistent therapeutic outcomes.

In response to the demand for more efficient and potent stimulation protocols, researchers developed theta burst stimulation (TBS), a patterned form of rTMS that applies short bursts of high-frequency pulses repeated at a theta rhythm (˜5 Hz). TBS, and in particular intermittent TBS (ITBS) has gained traction due to its ability to produce longer-lasting neuroplastic effects in a significantly shorter time than conventional rTMS. A further refinement of this approach is quadri-pulse stimulation (QPS) where bursts of four magnetic pulses are delivered in a regular pattern. When combined with the rhythmic structure of TBS, QPS forms the basis for quadri-pulse theta burst stimulation (QPS-TBS), which shows considerable promise in enhancing cortical plasticity and improving therapeutic outcomes in various clinical applications.

Despite the advantages offered by TBS and QPS protocols, a major limitation persists i.e. the stimulation patterns are typically standardized rather than individualized. However, the human cortex does not function uniformly across all individuals. A key component of cortical excitability is i-wave periodicity which refers to the rhythmic discharges that occur in the motor cortex. These i-waves are thought to reflect the activity of interneuronal networks that shape the response of pyramidal neurons and can vary substantially between individuals. Existing stimulation protocols which ignore these personalized neurophysiological patterns may therefore be suboptimal, potentially missing the critical timing windows necessary to most effectively induce neuroplastic changes.

The limitation underscores a pressing need for a solution that utilizes TMS protocols that are tailored to the individual neurophysiological profile of each patient. Specifically, by aligning the timing of stimulation pulses with the patient's unique i-wave periodicity, the solution provides more precisely modulated cortical circuits and enhance the efficacy of TMS-based interventions. Personalized protocols that adapt to individual i-wave characteristics could significantly reduce variability in treatment response and optimize the therapeutic impact for each patient.

### SUMMARY

The present invention relates to a personalized brain stimulation apparatus configured to enhance treatment of neurological and psychiatric disorders through i-wave periodicity-dependent Quadri-Pulse Theta Burst Stimulation (iQPS). The apparatus leverages individualized cortical response timing to optimize transcranial magnetic stimulation (TMS) parameters for each patient. The apparatus utilizes a TMS stimulator for delivering test pulses to a target motor cortical area and therapeutic theta bursts to a treatment region. An integrated electromyography (EMG) device records motor evoked potentials (MEPs) from for example an abductor pollicis brevis muscle and analyzes the timing between MEPs to determine the i-wave periodicity. By aligning stimulation timing with intrinsic neural activity, the apparatus promotes improved cortical plasticity and therapeutic efficacy. The invention is applicable to a range of conditions including depression, schizophrenia, stroke rehabilitation and neuropathic pain, providing a non-invasive, adaptive and patient-specific neuromodulation approach.

In an embodiment of the present invention, the invention discloses an apparatus for treating a neurological or psychiatric disorder in a patient using i-wave periodicity-dependent Quadri-Pulse Theta Burst Stimulation (iQPS). The apparatus comprising a transcranial magnetic stimulation (TMS) stimulator configured to deliver test pulses to a target motor cortical area of the patient and deliver theta bursts to a target treatment area with pulse frequency adjusted based on a patient's i-wave periodicity. The apparatus also includes an electromyography (EMG) device configured to record motor evoked potentials (MEPs) from a target muscle and analyze MEPs to determine the i-wave periodicity for adjusting the TMS pulse frequency. The test pulses are delivered to the motor cortex using TMS simulator, MEPs are recorded from the target muscle via the EMG device and the i-wave periodicity is derived from MEP intervals to adjust theta burst frequency for personalized therapy.

In one of the embodiments of the present invention, the target motor cortical area is the hand motor area of the patient.

In one of the embodiments of the present invention, the target muscle is the patient's abductor pollicis brevis muscle.

In one of the embodiments of the present invention, each of the theta burst is delivered by the TMS stimulator consists of four magnetic pulses.

In one of the embodiments of the present invention, the EMG device records MEPs from the abductor pollicis brevis (APB) muscle.

In one of the embodiments of the present invention, the neurological or psychiatric disorder includes but not limited to depression, schizophrenia, stroke, neuropathic pain.

In another embodiment of the present invention, the invention discloses a method for treating a neurological or psychiatric disorder in a patient using i-wave periodicity-dependent Quadri-Pulse Theta Burst Stimulation (iQPS). The method comprises delivering of test pulses via transcranial magnetic stimulation (TMS) to a target motor cortical area of the patient and recording of motor evoked potentials (MEPs) from a target muscle that contract in response to the stimulation of the target motor cortical area. The method also includes analyses of the recorded MEPs to determine the temporal intervals between consecutive MEPs and determining the patient's i-wave periodicity. In addition, quadri-pulse theta bursts are delivered via TMS to a treatment area with pulse frequency tailored to the patient's i-wave periodicity.

In one of the embodiments of the present invention, the target motor cortical area is the patient's hand motor area.

In one of the embodiments of the present invention, electromyography (EMG) electrodes are placed over the target motor hand area during test pulse delivery.

In one of the embodiments of the present invention, the target muscle is the abductor pollicis brevis (APB) muscle.

In one of the embodiments of the present invention, the neurological or psychiatric disorder includes but not limited to depression, schizophrenia, stroke, neuropathic pain.

In one of the embodiments of the present invention, the pulse frequency of each quadri-pulse theta burst is dynamically selected to match the individual's measured i-wave periodicity.

In one embodiment of the present invention, the pulse frequency of each quadri-pulse theta burst is selected to correspond to an assumed i-wave periodicity of the subject.

An apparatus for treating a neurological or psychiatric disorder in a patient using i-wave periodicity-dependent Quadri-Pulse Theta Burst Stimulation (IQPS), the apparatus comprising a transcranial magnetic stimulation (TMS) stimulator configured to deliver test pulses to a target motor cortical area in the patient and deliver theta bursts to a target treatment area in the patient, wherein each theta burst comprising four pulses having a pulse frequency, and wherein the pulse frequency is adjusted based on the patient's i-wave periodicity, and wherein the pulse frequency of each quadri-pulse theta burst is selected to correspond to an assumed i-wave periodicity. In the embodiment, the target motor cortical area is the hand motor area of the patient. In the embodiment, the assumed i-wave periodicity is a rhythm with equal intervals between the pulses or irregular intervals between the pulses. In the embodiment, neurological or psychiatric disorders include depression, schizophrenia, stroke, neuropathic pain. In the embodiment, the EM G measurement is not necessary.

For further clarification of the features and other embodiments of the invention, a more particular description is provided that will further explain the features and advantage of the invention with the illustration or the drawings. As will be appreciated, other embodiments of the present invention are possible utilizing, alone or in combination, one or more of the features set forth above or described in detail below.

### BRIEF DESCRIPTION OF THE DRAWINGS

A full understanding of the invention can be gained from the following description of the preferred embodiments when read in conjunction with the accompanying drawings in which:

FIG. 1 illustrates a block diagram of overall components of i-wave periodicity-dependent Quadri-Pulse Theta Burst Stimulation (iQPS) apparatus, in according to embodiments of the present invention disclosed herein.

FIG. 2 illustrates a method flow for treating neurological or psychiatric disorders in a patient using personalized i-wave periodicity-dependent Quadri-Pulse Theta Burst Stimulation (iQPS), in according to embodiments of the present invention disclosed herein.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION

References will now be made in detail to the presently preferred embodiments of the invention, examples of which are illustrated in the accompanying drawings. Throughout the following detailed description, the same reference numerals refer to the same elements in all figures.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and it is not intended to be limiting the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In the following description, reference will be made to the accompanying drawing, in which comparable functional elements are designated with like numerals. The aforementioned accompanying drawings show by way of illustration and not by the way of limitation, specific aspects and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in limited sense. It is noted that description herein is not intended as an extensive overview, and as such, concepts may be simplified in the interests of clarity and brevity. All documents mentioned in this application are hereby incorporated by reference in their entirety.

For the purpose of this specification, the following definitions shall apply:
TMS-TMS is a noninvasive procedure that uses magnetic fields to stimulate nerve cells in the brain to improve symptoms of neurological or psychiatric disorders. Existing devices and methods for TMS are known and described in the art (see, e.g., Lefaucheur, J-P. et al. “Evidence-based guidelines on the therapeutic use of repetitive transcranial magnetic stimulation (rTMS): An update (2014-2018).” Clin. Neurophysiol. 131 (2): 474-528 (2020); Burke, M. J., Fried P. J., & Pascual-Leone A. “Transcranial magnetic stimulation: Neurophysiological and clinical applications.” Handb. Clin. Neurol. 163:73-92 (2019); Vucic S. et al. “Clinical diagnostic utility of transcranial magnetic stimulation in neurological disorders. Updated report of an IFCN committee.” Clin. Neurophysiol. 150:131-175 (2023); Lefaucheur J. P. “Transcranial magnetic stimulation.” Handb. Clin. Neurol. 160:559-580 (2019); Iglesias A. H. “Transcranial magnetic stimulation as treatment in multiple neurologic conditions.” Curr. Neurol. Neurosci. Rep. 20 (1): 1 (2020); Gogulski J. et al. “Personalized repetitive transcranial magnetic stimulation for depression.” Biol. Psychiatry Cogn. Neurosci. Neuroimaging. 8 (4): 351-360 (2023); and Jannati A. et al. “Assessing the mechanisms of brain plasticity by transcranial magnetic stimulation.” Neuropsychopharmacology. 48 (1): 191-208 (2023); Richter K., Kellner S., & Licht C. “rTMS in mental health disorders.” Front. Netw. Physiol. 3:943223 (2023)).

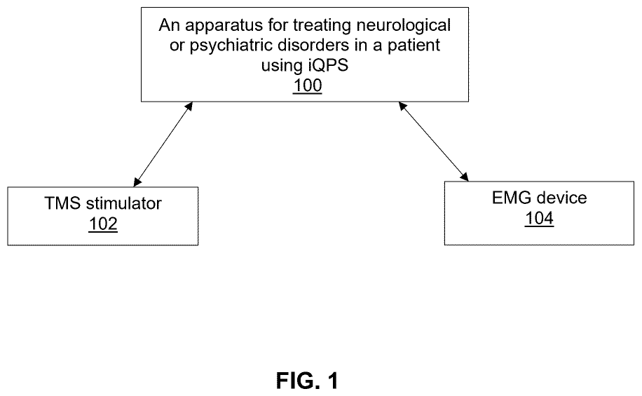

Referring to FIG. 1, an apparatus for treating neurological or psychiatric disorders in a patient using personalized i-wave periodicity-dependent Quadri-Pulse Theta Burst Stimulation (iQPS) is disclosed. The apparatus 100 comprises a TMS stimulator 102 and an EM G device 104. The TMS stimulator 102 is configured to deliver both test pulses and therapeutic theta bursts to specified regions of the brain. Specifically, the TMS stimulator 102 delivers test pulses to a target motor cortical area of the patient to evoke neural responses and deliver therapeutic theta bursts to a designated treatment area. The pulse frequency of the theta bursts is adjustable and personalized based on the patient's measured i-wave periodicity. Each theta burst consists of four magnetic pulses forming a quadri-pulse sequence optimized to modulate cortical excitability and promote neuroplastic changes in the brain. The EMG device 104 is operably connected to record motor evoked potentials (MEPs) from a target muscle typically one that contracts in response to stimulation of the motor cortex. In addition, the motor cortical area is the hand motor area and the target muscle is the abductor pollicis brevis (APB) muscle in the patient's hand. The EMG device 104 captures the MEP waveforms during the test pulse phase and analyzes the temporal intervals between consecutive MEPs. This analysis yields the patient's i-wave periodicity i.e. a neurophysiological marker reflecting the timing of indirect waves generated in the motor cortex by TMS. In addition, the test pulses are delivered via the TMS stimulator 102 to the motor cortex to evoke MEPs. The EMG device 104 records these responses from the APB muscle and software within the apparatus analyzes the inter-MEP intervals to compute i-wave periodicity. Once the value is obtained, the TMS stimulator 102 transitions to treatment mode, delivering quadri-pulse theta bursts at a frequency tailored to the patient's specific i-wave timing. Further, individual variability in i-wave periodicity significantly influences cortical plasticity and response to TMS, therefore, tailoring the stimulation parameters based on the measurements allows for improved clinical outcomes.

In one of the embodiments of the present invention, each theta burst delivered by the TMS stimulator consists of four magnetic pulses.

In one of the embodiments of the present invention, the EM G device records MEPs from the abductor pollicis brevis (APB) muscle.

In one of the embodiments of the present invention, the invention provides several advantages over conventional TMS therapy methods. The invention enables a high degree of personalization by tailoring stimulation parameters to the individual neurophysiological characteristics of each patient, specifically their i-wave periodicity, thereby achieving more precise and effective treatment. The personalized nature of iQPS improves overall therapy effectiveness as it accounts for individual cortical dynamics, resulting in better clinical outcomes compared to standard, one-size-fits-all TMS protocols. Moreover, iQPS is capable of inducing long-lasting changes in cortical plasticity, extending therapeutic effects well beyond the immediate period of stimulation. By reducing the likelihood of adverse effects through individualized stimulation, the approach enhances the safety and tolerability of therapy. The method is applicable to a wide spectrum of neurological and psychiatric disorders such as depression, schizophrenia, stroke and neuropathic pain, making it a versatile solution for diverse patient populations. The embodiment is also technically feasible using current TMS and EMG systems, allowing easy adoption in clinical settings. Ultimately, by optimizing therapeutic effects and minimizing side effects, iQPS contributes to significant long-term improvements in neurological recovery and patient quality of life.

In one of the embodiments of the present invention, the apparatus is used to treat various neurological and psychiatric conditions. In particular, it is suitable for disorders such as depression, schizophrenia, stroke-related motor deficits and neuropathic pain. The conditions often involve disruptions in cortical connectivity and plasticity which can be addressed by modulating neural circuits through targeted TMS. For example, in patients recovering from stroke, personalized iQPS may help promote functional reorganization of the motor cortex. In psychiatric conditions such as depression, theta burst stimulation has been shown to influence prefrontal cortical networks involved in mood regulation.

In one of the embodiments of the present invention, the apparatus may further be implemented with user interfaces and control software that allow clinicians to input patient data, view real-time MEP recordings and automatically calculate stimulation parameters. The entire setup is specialized and integrated to perform iQPS accurately and consistently across treatment sessions.

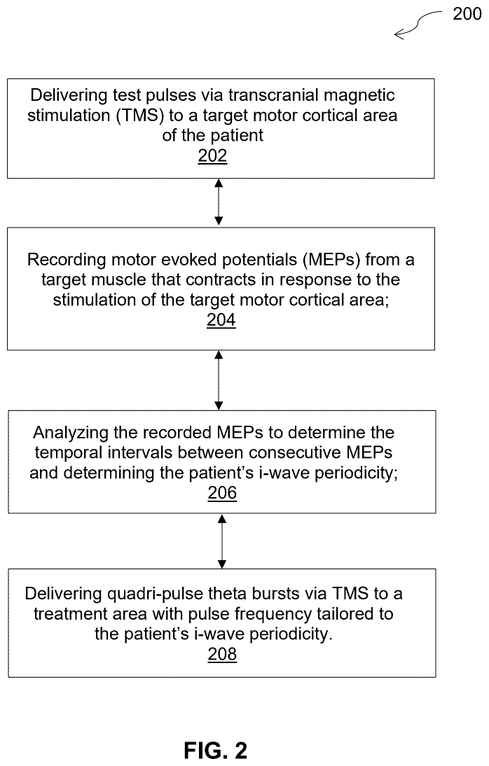

Referring to FIG. 2, the invention discloses a method 200 for treating neurological or psychiatric disorders in a patient using i-wave periodicity-dependent Quadri-Pulse Theta Burst Stimulation (iQPS). The method 200 is configured to enhance cortical plasticity by aligning magnetic stimulation patterns with the patient's intrinsic cortical timing, specifically the i-wave periodicity. In the embodiment, according to step 202, the method 200 discloses delivery of test pulses via TMS to a target motor cortical area, typically the hand motor area which is known to reliably elicit motor responses. The test pulses are used to evoke motor evoked potentials (MEPs) which represent the electrical responses of muscles following stimulation of the motor cortex. In addition, in step 204, the MEPs are recorded using electromyographic (EMG) electrodes which are placed over a corresponding target muscle commonly known as abductor pollicis brevis (APB) muscle of the hand. The EMG electrodes capture the electrical activity generated by muscle activity in response to cortical stimulation. This step is critical as it provides the raw data required to identify the rhythmic patterns of indirect corticospinal volleys known as i-waves. In the embodiment, according to step 206, the method 200 includes analyzing the recorded MEPs to determine the temporal intervals between consecutive peaks in the MEP waveform. The intervals reflect the patient's i-wave periodicity which varies among individuals and is influenced by factors such as cortical excitability and synaptic efficiency. The analysis may be performed using automated signal processing algorithms capable of identifying distinct MEP components and calculating time intervals with millisecond precision. The step provides a neurophysiological biomarker that enables the personalization of subsequent TMS therapy by ensuring that external stimulation is synchronized with internal cortical timing. Additionally, in step 208, quadri-pulse theta bursts are delivered to a treatment area using the TMS device. Each theta burst consists of four high-frequency magnetic pulses. The pulse frequency of these bursts is dynamically adjusted to match the individual's measured i-wave periodicity. By synchronizing stimulation with intrinsic cortical timing, the method maximizes the potential for inducing long-term potentiation (LTP) or long-term depression (LTD) thereby facilitating neuroplastic changes within the brain. The treatment area may vary depending on the disorder i.e. for depression or schizophrenia, the prefrontal cortex may be targeted, for stroke rehabilitation or neuropathic pain and stimulation is directed toward the motor cortex or relevant sensorimotor areas.

In one of the embodiments of the present invention, the neurological or psychiatric disorder includes but not limited to depression, schizophrenia, stroke, neuropathic pain.

In one of the embodiments of the present invention, stimulation can include both monophasic and biphasic pulses with pulse duration and direction being flexibly adjustable to the patient's needs. This enables individualized therapy tailored to the specific neurophysiological characteristics of each patient.

In one of the embodiments of the present invention, the apparatus is particularly well-suited for treating neurological and psychiatric disorders characterized by altered cortical connectivity or impaired plasticity. For example, in major depressive disorder, theta burst stimulation of the dorsolateral prefrontal cortex can help restore function in mood-regulating networks. In stroke patients, personalized iQPS to the lesioned motor cortex may promote reorganization of motor pathways, enhancing motor recovery. In cases of neuropathic pain, synchronized stimulation may help normalize maladaptive plasticity in sensory circuits. The inclusion of i-wave periodicity in treatment planning represents a significant advancement over conventional TMS which typically employs fixed stimulation parameters that fail to account for inter-individual variability in cortical response.

In one of the embodiments of the present invention, any suitable computer useable medium may be utilized for software aspects of the invention. The computer-usable or computer-readable medium may be for example but not limited to an electronic, magnetic, optical, electromagnetic, infrared, or semiconductor system, apparatus, device, or propagation medium. The computer readable medium may include transitory and/or non-transitory embodiments. More specific embodiments (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Further, computer-usable or computer-readable medium could even be paper or another suitable medium upon which the program is printed as the program can be electronically captured via for instance, optical scanning of the paper or other medium, then compiled, interpreted or otherwise processed in a suitable manner, if necessary and then stored in a computer memory. In addition, a computer-usable or computer-readable medium may be any medium that can contain, store, communicate, propagate or transport the program for use by or in connection with the instruction execution system, apparatus, or device.

In one of the embodiments of the present invention, the program code used for carrying out operations of the invention may be written in an object oriented programming language such as Java, Smalltalk, C++ or similar language. The program code for carrying out operations of the invention may also be written in conventional procedural programming languages such as the C programming language or similar programming languages. The program code may be executed by a processor, (application specific integrated circuit ASIC) or other component that executes the program code. The program code may be simply referred to as a software application that is stored in memory (such as the computer readable medium discussed above). The program code may cause the processor (processor-controlled device) to produce a graphical user interface (GUI) that is visually produced on a display device. The program code, however, may operate in any processor-controlled device such as a computer, server, personal digital assistant, phone, television, or any processor controlled device utilizing the processor and/or a digital signal processor. The program code may locally and/or remotely execute. The program code for example may be entirely or partially stored, accessed and downloaded in local memory of the processor-controlled device. A user's computer for example may entirely execute the program code or only partly execute the program code. Further, the program code may be a stand-alone software package that is at least partly executed on the user's computer or partly executed on a remote computer or entirely on a remote computer or server.

In one of the embodiments of the present invention, the invention may utilize the communications network which includes a cable network operating in the radio-frequency domain and/or the Internet Protocol (IP) domain. The communications network, however, may also include a distributed computing network such as the Internet (sometimes alternatively known as the “World Wide Web”), an intranet, a local-area network (LAN), and/or a wide-area network (WAN). The communications network may also include coaxial cables, copper wires, fiber optic lines, and/or hybrid-coaxial lines. The communications network includes wireless portions utilizing any portion of the electromagnetic spectrum and any signaling standard (such as the IEEE 802 family of standards, GSM/CDMA/TDMA or any cellular standard, and/or the ISM band). The communications network may even include powerline portions in which signals are communicated via electrical wiring. In some aspects, wireless communication interfaces may include but are not limited to, an Intranet connection, Internet, Personal Area Networks (PANs) for the exchange of data over short distances, e.g., using short-wavelength radio transmissions in the industrial, scientific, and medical (ISM) band ISM band from 2400-2480 MHz) from fixed and mobile devices (e.g., Bluetooth® technology), wireless fidelity (Wi-Fi), Wi-Max, IEEE 802.11 technology, radio frequency (RF), Infrared Data Association (IrDA) compatible protocols, Local Area Networks (LANs), Wide Area Networks (WANs), Shared Wireless Access Protocol (SWAP), Zigbee, Near-Field Communication (NFC), LiFi, 5G, any combinations thereof, and other types of wireless networking protocols.

It should be understood that the examples provided herein are intended only for purposes of illustration and any number of other implementations is also contemplated. Additionally, the referenced examples (including the described rules and/or other techniques) can be combined in any number of ways.

Although an overview of the inventive subject matter has been described with reference to specific example implementations, various modifications and changes can be made to those implementations without departing from the broader scopes of implementation of the present disclosure. Such implementation of the inventive subject matter can be referred to herein, individually or collectively, by the term “invention” merely for convenience without intending to voluntarily limit the scope of this application to any single disclosure or inventive concept if more than one is, in fact is disclosed.

The implementations illustrated herein are described in sufficient detail to enable those skilled in the art to practice the teachings disclosed. Other implementations can be used and derived therefrom, such that structural substitutions and changes can be made without departing from the scope of this disclosure. The Detailed Description, therefore, is not to be taken in a limiting sense, and the scope of various implementations is defined only by the appended claims, along with the full range of equivalents to which such claims are entitled.

As used herein, the term “or” can be construed in either an inclusive or exclusive sense. Moreover, plural instances can be provided for resources or structures described herein as a single instance. These and other variations, modifications, additions, and improvements fall within a scope of implementations of the present disclosure as represented by the appended claims. The specification and drawings are, accordingly, to be regarded in an illustrative rather than a restrictive sense.

What is claimed is:

1. An apparatus for treating a neurological or psychiatric disorder in a patient using i-wave periodicity-dependent Quadri-Pulse Theta Burst Stimulation (iQPS), the apparatus comprising:
a transcranial magnetic stimulation (TMS) stimulator configured to:
deliver test pulses to a target motor cortical area in the patient; and
deliver theta bursts to a target treatment area in the patient,
wherein each theta burst comprising four pulses having a pulse frequency, and wherein the pulse frequency is adjusted based on the patient's i-wave periodicity; and
an electromyography (EMG) device configured to:
record motor evoked potentials (MEPs) from a target muscle, the target muscle contracts in response to stimulation of the target motor cortical area; and
analyze the MEPs to determine the patient's i-wave periodicity.

2. The apparatus as claimed in claim 1, wherein the target motor cortical area is the hand motor area of the patient.

3. The apparatus as claimed in claim 1, wherein the target muscle is the patient's abductor pollicis brevis muscle.

4. The apparatus as claimed in claim 1, wherein the patient's i-wave periodicity is temporal intervals between consecutive MEPs.

5. The apparatus as claimed in claim 1, wherein neurological or psychiatric disorder include depression, schizophrenia, stroke, neuropathic pain.

6. The apparatus as claimed in claim 1, wherein the pulse frequency within the theta burst is constant.

7. A method for treating a neurological or psychiatric disorder in a patient using i-wave periodicity-dependent Quadri-Pulse Theta Burst Stimulation (iQPS), the method comprising:
delivering test pulses via transcranial magnetic stimulation (TMS) to a target motor cortical area of the patient;
recording motor evoked potentials (MEPs) from a target muscle that contracts in response to the stimulation of the target motor cortical area;
analyzing the recorded MEPs to determine the temporal intervals between consecutive MEPs and determining the patient's i-wave periodicity; and
delivering quadri-pulse theta bursts via TMS to a treatment area with pulse frequency tailored to the patient's i-wave periodicity.

8. The method of claim 7, wherein the target motor cortical area is the patient's hand motor area.

9. The method as claimed in claim 7, wherein electromyographic (EMG) electrodes are placed over the target motor hand area during test pulse delivery.

10. The method as claimed in claim 7, wherein the target muscle is the abductor pollicis brevis (APB) muscle.

11. The method as claimed in claim 7, wherein the neurological or psychiatric disorder includes depression, schizophrenia, stroke, neuropathic pain.

12. The method as claimed in claim 7, wherein the pulse frequency of each quadri-pulse theta burst is dynamically selected to match the individual's measured i-wave periodicity.

13. A n apparatus for treating a neurological or psychiatric disorder in a patient using i-wave periodicity-dependent Quadri-Pulse Theta Burst Stimulation (iQPS), the apparatus comprising:
a transcranial magnetic stimulation (TMS) stimulator configured to:
deliver test pulses to a target motor cortical area in the patient; and
deliver theta bursts to a target treatment area in the patient,
wherein each theta burst comprising four pulses having a pulse frequency, and wherein the pulse frequency is adjusted based on the patient's i-wave periodicity; and
wherein the pulse frequency of each quadri-pulse theta burst is selected to correspond to an assumed i-wave periodicity.

14. The apparatus as claimed in claim 13, wherein the target motor cortical area is the hand motor area of the patient.

15. The apparatus as claimed in claim 13, wherein the assumed i-wave periodicity is a rhythm with equal intervals between the pulses or irregular intervals between the pulses.

16. The apparatus as claimed in claim 13, wherein neurological or psychiatric disorders include depression, schizophrenia, stroke, neuropathic pain.
